(19) United States
(12) Patent Application Publication
(10) Pub. No.: US 2020/0000624 A1
(43) Pub. Date: Jan. 2, 2020

(54) Ostomy Leakage Alert System

(71) Applicants: Jennifer Gibbons, Herriman, UT (US); Sarah Gibbons, Herriman, UT (US)

(72) Inventors: Jennifer Gibbons, Herriman, UT (US); Sarah Gibbons, Herriman, UT (US)

(21) Appl. No.: 16/021,192

(22) Filed: Jun. 28, 2018

(51) Int. Cl. A61F 5/445; A61B 5/00; A61F 5/44

(52) U.S. Cl. CPC A61F 5/445; A61F 5/443; A61F 5/4404; A61B 5/746

(57) ABSTRACT

An ostomy leakage alert system for notifying a user of ostomy leakage includes a sensor assembly comprising a sensor pad and a sensor integrally disposed within the sensor pad. The sensor pad has an aperture to couple with an ostomy bag and an ostomy skin barrier disc. A lead has a sensor end coupled to the sensor and a connector end having a male connector. An alarm assembly is coupled to the lead, the alarm assembly comprising an alarm housing, a battery, a CPU, an alarm speaker, a female port, and an N-channel. The female port selectively receives the male connector of the lead. The battery, the CPU, the alarm speaker, the female port, and the N-channel are disposed within the alarm housing. The sensor assembly is configured to detect a leakage from the ostomy bag and to activate the alarm assembly, notifying a wearer of the leakage.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to leak detection devices and more particularly pertains to a new leak detection device for notifying a user of ostomy leakage.

### BRIEF SUMMARY OF THE INVENTION

An embodiment of the disclosure meets the needs presented above by generally comprising a sensor assembly comprising a sensor pad and a sensor, the sensor being integrally disposed within the sensor pad. The sensor pad has an aperture that is configured to couple with an ostomy bag and an ostomy skin barrier disc. A lead has a sensor end coupled to the sensor at an edge of the sensor pad and a connector end having a male connector. An alarm assembly is coupled to the lead, the alarm assembly comprising an alarm housing, a battery, a CPU, an alarm speaker, a female port, and an N-channel. The female port selectively receives the male connector of the lead. The battery, the CPU, the alarm speaker, the female port, and the N-channel are disposed within the alarm housing. The CPU is in operational communication with the battery, the alarm speaker, the female port, and the N-channel. The sensor assembly is configured to detect a leakage from the ostomy bag and to activate the alarm assembly, notifying a wearer of the leakage.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of an ostomy leakage alert system according to an embodiment of the disclosure.

FIG. 2 is a front elevation view of an embodiment of the disclosure.

FIG. 3 is a top plan view of an embodiment of the disclosure.

FIG. 4 is a side elevation view of an embodiment of the disclosure.

FIG. 5 is a rear elevation view of an embodiment of the disclosure and an ostomy bag.

FIG. 6 is an in-use view of an embodiment of the disclosure.

FIG. 7 is a cross-sectional view of an embodiment of the disclosure along line 7-7 of FIG. 4.

### DETAILED DESCRIPTION OF THE INVENTION

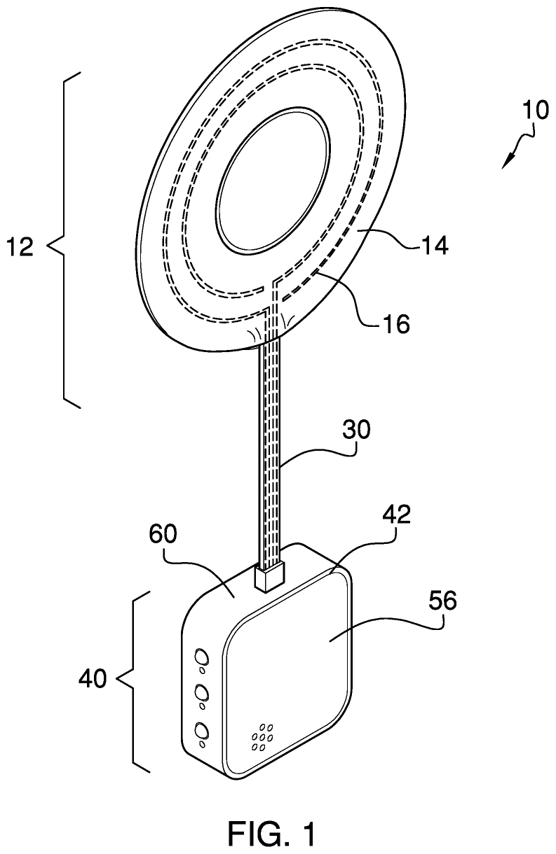

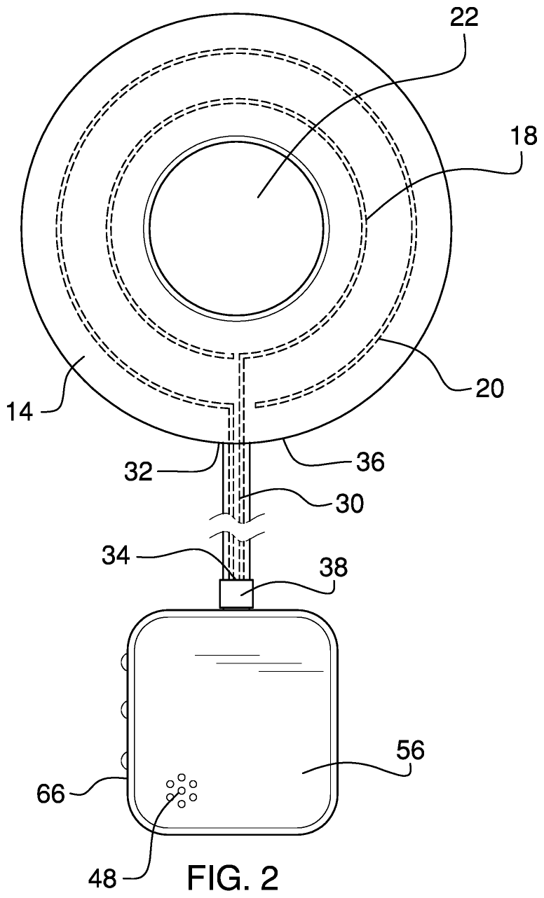

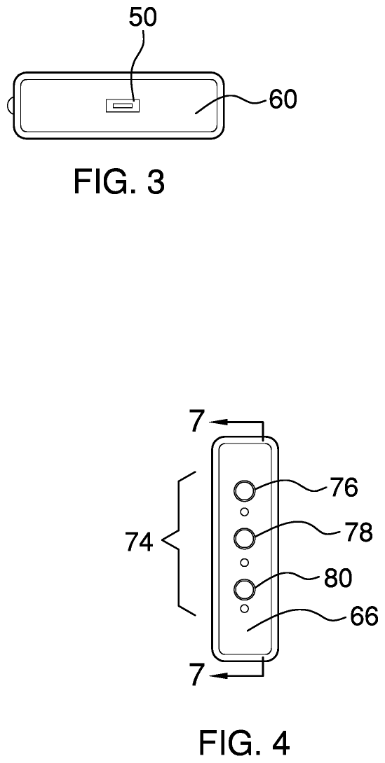

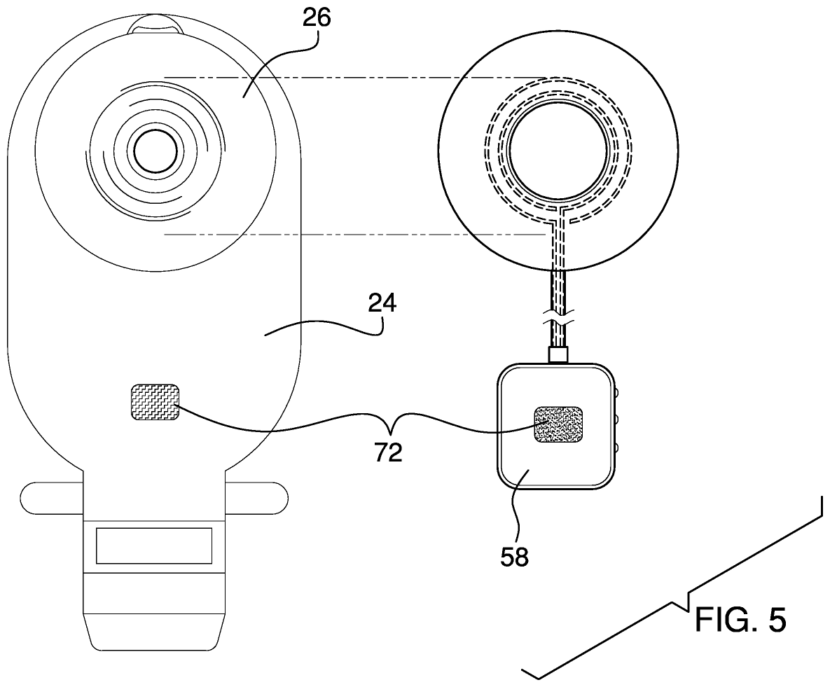

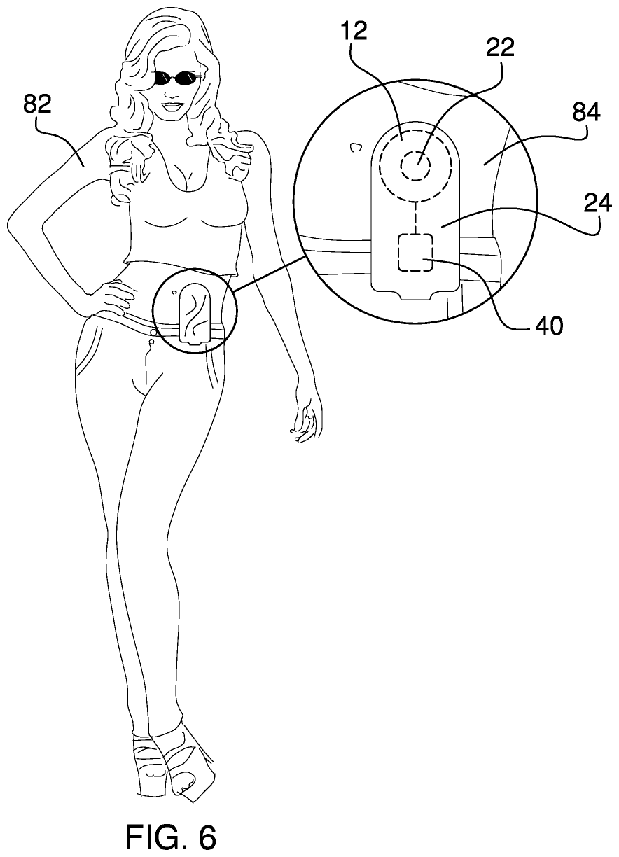

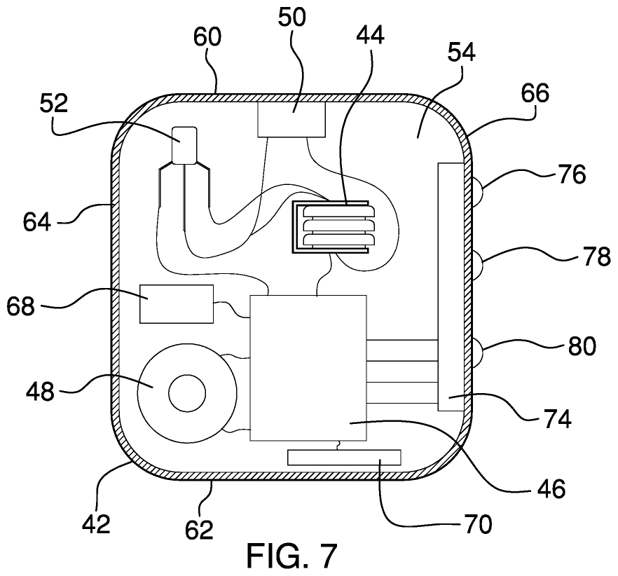

With reference now to the drawings, and in particular to FIGS. 1 through 7 thereof, a new leak detection device embodying the principles and concepts of an embodiment of the disclosure and generally designated by the reference numeral 10 will be described.

As best illustrated in FIGS. 1 through 7, the ostomy leakage alert system 10 generally comprises a sensor assembly 12, the sensor assembly comprising a sensor pad 14 and a sensor 16 integrally disposed within the sensor pad. The sensor comprises a first pair 18 and a second pair of conductive concentric trace rings 20, the second pair of trace rings circumscribing the first pair of trace rings. The sensor pad 14 has an aperture 22 that is configured to couple with an ostomy bag 24 and an ostomy skin barrier disc 26. The sensor pad may be adhesive. The aperture may be a variety of shapes or may be customizable to fit with any ostomy bag and skin barrier disc. A lead 30 has a sensor end 32 and a connector end 34. The sensor end 32 is coupled to the sensor 16 at an edge 36 of the sensor pad 14. The connector end 34 has a male connector 38.

An alarm assembly 40 is coupled to the lead 30. The alarm assembly comprises an alarm housing 42, a battery 44, a CPU 46, an alarm speaker 48, a female port 50, and an N-channel 52. The female port 50 selectively receives the male connector 38 of the sensor assembly. The alarm housing 42 has cavity 54 between a front side 56, a rear side 58, a top side 60, a bottom side 62, a left side 64, and a right side 66. The battery 44, the CPU 46, the alarm speaker 48, and the N-channel 52 are coupled within the cavity 54 of the alarm housing. The female port 50 is disposed within the top side 60 of the alarm housing through to the cavity 54. The N-channel is in operational communication with the CPU, the battery, and the female port. A vibration mechanism 68 and a wireless transmitter 70 may also be coupled within the cavity 54 of the alarm housing. The wireless transmitter may be a Bluetooth or Wi-Fi transmitter and is configured to connect to a cellphone. The CPU 46 is in operational communication with the battery 44, the alarm speaker 48, the female port 50, the N-channel 52, the vibration mechanism 68, and the wireless transmitter 70. The alarm speaker may be piezoelectric. A hook-and-loop fastener 72 is coupled to the rear side 58 of the alarm housing and to the ostomy bag 24 to secure the alarm housing to the ostomy bag.

A plurality of controls 74 is disposed through the right side 66 of the alarm housing to the cavity 54. The plurality of controls may comprise a power button 76, an option button 78, and an SOS button 80, each of which is in operational communication with the CPU. The power button 76 turns the alarm assembly 40 on and off. The option button 78 cycles between a plurality of alert methods. The plurality of alert methods may comprise only the alarm speaker 48, only the vibration mechanism 68, both the alarm speaker and the vibration mechanism, and the wireless transmitter 70. The SOS button 80 sends a signal through the wireless transmitter.

In use, a wearer 82 applies the sensor pad 14 directly to her skin 84 with the aperture 22 around her stoma and the lead 30 oriented downwards. The ostomy skin barrier disc 26 and bag 24 are connected as normal but through the aperture 22 of the sensor pad. The lead 30 is connected to the alarm assembly 40, which is then turned on with the power button 76. The wearer selects the desired alert method for each of the first pair and the second pair of trace rings with the option button 78. If there is a leak from the stoma, the liquid will complete a circuit between the first pair of trace rings 18, signaling the CPU 46 to activate the chosen alert method. As the liquid spreads, the second pair of trace rings 20 sends a second signal to the CPU to activate the chosen alert method. In case of an emergency situation, the wearer may use the SOS button 80 to send an emergency call or message through the connected cellphone.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An ostomy leakage alert system, the system comprising:
a sensor assembly, the sensor assembly comprising a sensor pad and a sensor, the sensor being integrally disposed within the sensor pad, the sensor pad having an aperture, wherein the aperture in the sensor pad is configured to couple with an ostomy bag and an ostomy skin barrier disc;
a lead having a sensor end coupled to the sensor at an edge of the sensor pad and a connector end having a male connector;
an alarm assembly coupled to the lead, the alarm assembly comprising an alarm housing, a battery, a CPU, an alarm speaker, a female port, and an N-channel, the female port selectively receiving the male connector of the lead, the battery, the CPU, the alarm speaker, the female port, and the N-channel being disposed within the alarm housing, the CPU being in operational communication with the battery, the alarm speaker, the female port, and the N-channel;
wherein the sensor assembly is configured to detect a leakage from the ostomy bag and activate the alarm assembly, wherein the alarm assembly is configured to notify a wearer upon said leakage.

2. The ostomy leakage alert system of claim 1, further comprising said sensor comprising a first pair of conductive concentric trace rings, the first pair of trace rings sending a first signal to the CPU upon contact with liquid.

3. The ostomy leakage alert system of claim 2, further comprising said sensor having a second pair of conductive concentric trace rings, the second pair of trace rings circumscribing the first pair of trace rings, the second pair of trace rings sending a second signal to the CPU upon contact with liquid.

4. The ostomy leakage alert system of claim 1, further comprising said alarm housing having a front side separated from a rear side, a top side separated from a bottom side, and a left side separated from a right side, the alarm housing having a cavity between the front side, the rear side, the top side, the bottom side, the left side, and the right side, the battery, the CPU, the alarm speaker, and the N-channel being coupled within the cavity of the alarm housing, and the female port being disposed within the top side of the alarm housing through to the cavity.

5. The ostomy leakage alert system of claim 4, further comprising said alarm housing having a hook-and-loop fastener, the hook-and-loop fastener being coupled to the rear side, wherein the hook-and-loop fastener is configured to secure the alarm housing to the ostomy bag.

5. The ostomy leakage alert system of claim 4, further comprising a plurality of controls coupled to the alarm housing, the plurality of controls being disposed within the right side of the alarm housing through to the cavity, the plurality of controls being in operational communication with the CPU.

6. The ostomy leakage alert system of claim 5, further comprising a vibration mechanism and a wireless transmitter coupled within the cavity of the alarm housing, wherein the wireless transmitter is configured to connect to a cellphone.

7. The ostomy leakage alert system of claim 6, further comprising said plurality of controls comprising a power button and an option button, the power button being in operational communication with the CPU and the battery, the option button being in operational communication with the CPU, the option button cycling between a plurality of alert methods, the plurality of alert methods comprising only the alarm speaker, only the vibration mechanism, both the alarm speaker and the vibration mechanism, and the wireless transmitter.

8. The ostomy leakage alert system of claim 7, further comprising said plurality of controls comprising an SOS button, the SOS button being in operational communication with the CPU and the wireless transmitter, wherein the SOS button is configured to send an emergency call or message through the connected cellphone.

9. An ostomy leakage alert system, the system comprising:
a sensor assembly, the sensor assembly comprising a sensor pad and a sensor, the sensor being integrally disposed within the sensor pad, the sensor comprising a first pair and a second pair of conductive concentric trace rings, the second pair of trace rings circumscribing the first pair of trace rings, the sensor pad having an aperture, wherein the aperture in the sensor pad is configured to couple with an ostomy bag and an ostomy skin barrier disc;
a lead having a sensor end coupled to the sensor at an edge of the sensor pad and a connector end having a male connector;
an alarm assembly coupled to the lead, the alarm assembly comprising an alarm housing, a battery, a CPU, an alarm speaker, a female port, and an N-channel, the alarm housing having a front side separated from a rear side, a top side separated from a bottom side, and a left side separated from a right side, the alarm housing having a cavity between the front side, the rear side, the top side, the bottom side, the left side, and the right side, the battery, the CPU, the alarm speaker, and the N-channel being coupled within the cavity of the alarm housing, and the female port being disposed within the top side of the alarm housing through to the cavity, the CPU being in operational communication with the battery, the alarm speaker, the female port, and the N-channel, the female port selectively receiving the male connector of the sensor assembly;
a hook-and-loop fastener coupled to the alarm assembly, the hook-and-loop fastener being coupled to the rear side of the alarm housing, wherein the hook-and-loop fastener is configured to secure the alarm housing to the ostomy bag;
a vibration mechanism coupled within the cavity of the alarm housing, the vibration mechanism being in operational communication with the CPU and the battery;
a wireless transmitter coupled within the cavity of the alarm housing, the wireless transmitter being in operational communication with the CPU and the battery, wherein the wireless transmitter is configured to connect to a cellphone; and
a plurality of controls coupled to the alarm assembly, the plurality of controls being disposed through the right side of the alarm housing to the cavity, the plurality of controls comprising a power button, an option button, and an SOS button, the power button being in operational communication with the CPU and the battery, the option button being in operational communication with the CPU, the option button cycling between a plurality of alert methods, the plurality of alert methods comprising only the alarm speaker, only the vibration mechanism, both the alarm speaker and the vibration mechanism, and the wireless transmitter, an SOS button, the SOS button being in operational communication with the CPU and the wireless transmitter, wherein the SOS button is configured to send an emergency call or message through the connected cellphone;
wherein the first pair of trace rings sends a first signal to the CPU upon contact with liquid from leakage and the second pair of trace rings sends a second signal to the CPU upon contact with liquid from leakage, each of the first signal and the second signal activating an alert method of the plurality of alert methods to alert a wearer to the leakage.
